(19) United States
(12) Patent Application Publication
(10) Pub. No.: US 2023/0289537 A1
(43) Pub. Date: Sep. 14, 2023

(54) SYSTEM FOR RENDERING ELECTRONIC MEDICAL RECORD DATA AND LANGUAGE INTERPRETATION DATA ON DISPARATE DEVICES AT A HEALTHCARE PROVIDER LOCATION

(71) Applicant: Language Line Services, Inc., Monterey, CA (US)

(72) Inventors: Lindsay D'Penha, Carmel, CA (US); Jeffrey Cordell, Carmel, CA (US); James Boutcher, Carmel, CA (US)

(73) Assignee: Language Line Services, Inc., Monterey, CA (US)

(21) Appl. No.: 17/693,235

(22) Filed: Mar. 11, 2022

(51) Int. Cl. G06F 40/58; G16H 10/60; G06F 3/0482

(52) U.S. Cl. CPC G06F 40/58; G16H 10/60; G06F 3/0482

(57) ABSTRACT

A computer-implemented process computer-implemented process renders, at an EMR device positioned at a healthcare provider location, an EMR user interface to access EMR data of a patient. Furthermore, the computer-implemented process receives, via a language interpretation request indicum of the EMR user interface, a request for language interpretation. The computer-implemented process sends, from the EMR device to an EMR server, the EMR data of the patient. Additionally, the computer-implemented process receivers, from the EMR server at the EMR device, a visual code indicium. Finally, the computer-implemented process renders, at the EMR device, the visual code indicium to link the EMR device with a language interpretation device also positioned at the healthcare provider location such that the language interpretation device renders a language interpretation session between a healthcare provider and the patient without being rendered by the EMR device.

### BACKGROUND

### 1. Field

This disclosure generally relates to the field of language interpretation. More particularly, the disclosure relates to computer-implemented language interpretation systems for language interpretation at a healthcare provider location.

### 2. General Background

Healthcare providers (e.g., doctors, nurses, physician's assistants, dentists, etc.) are becoming increasingly reliant on the use of electronic medical record (“EMR”) systems to manage patients' medical data. For instance, an EMR may store information such as a patient's medical history, dates of office visits, prescription drug information, known allergies, and emergency contact information. The EMR, itself, may have an EMR identifier that may be used to search for the EMR.

Accordingly, when a patient visits a healthcare provider, the healthcare provider will often use a computing device (e.g., tablet device, desktop computer, etc.) to access the EMR corresponding to the patient during the patient's office visit.

An additional use for an EMR is to store information regarding the language(s) spoken by a patient. (The term “language” referred to herein is the method of voice-based communication traditionally used by a group of people originating from a particular geographical location, country, or region, but may also include non-voice-based communication such as sign language or Braille.) Conventional computing systems used by the healthcare provider may allow the healthcare provider to initiate a request for a human language interpreter if the patient is a limited English proficiency speaker (“LEP”). A language interpretation session may then be established via the healthcare provider's computing device, whereby a language interpreter virtually translates the communications between the healthcare provider and the LEP.

However, use of the healthcare provider's computing device to provide both EMR data access and a language interpretation session has led to significant user interface problems for the healthcare provider. Specifically, the healthcare provider typically relies on the associated computing device (e.g., tablet device) to make notes and update records in the EMR system. For the healthcare provider to have to routinely pause such device interaction in order to allow the LEP to view and/or hear the language interpreter, the ability of the healthcare provider to provide an optimal quality of service is diminished.

As a result, conventional systems that provide for EMR data access/management and language interpretation have a user interface that is too cumbersome to use in an effective manner by healthcare providers.

### SUMMARY

In one embodiment, a computer-implemented process renders, at an EMR device positioned at a healthcare provider location, an EMR user interface to access EMR data of a patient. Furthermore, the computer-implemented process receives, via a language interpretation request indicum of the EMR user interface, a request for language interpretation. The computer-implemented process sends, from the EMR device to an EMR server, the EMR data of the patient. Additionally, the computer-implemented process receives, from the EMR server at the EMR device, a visual code indicium. Finally, the computer-implemented process renders, at the EMR device, the visual code to link the EMR device with a language interpretation device also positioned at the healthcare provider location such that the language interpretation device renders a language interpretation session between a healthcare provider and the patient without being rendered by the EMR device.

Alternatively, a computer program product may have a computer readable storage device with a computer readable program stored thereon that implements the functionality of the aforementioned processes. As yet another alternative, a system may implement the processes via various componentry.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1 illustrates a healthcare provider location (e.g., a doctor's office, health clinic, dentist's office, etc.) in which a healthcare provider provides in-person healthcare services to an LEP patient.

FIG. 2A illustrates an EMR user interface that may be rendered by the EMR device.

FIG. 2B illustrates an example of a QR code being displayed and a short form code by the ERM device for image capture by the language interpretation device.

FIG. 2C illustrate an alternative process in which the healthcare provider may link the EMR device.

FIG. 3A illustrates an example of how the EMR device is linked with a language interpretation device.

FIG. 3B illustrates the healthcare provider and the LEP patient participating in a language interpretation session via the language interpretation device.

FIG. 4 illustrates the EMR user interface of the EMR device rendering a language interpretation device menu of pre-linked language interpretation devices.

FIG. 5 illustrates a requestor/fulfiller configuration that links the EMR device with the language interpretation device.

FIG. 6 illustrates a system configuration for the requestor/fulfiller configuration illustrated in FIG. 5.

FIG. 7 illustrates a computer-implemented process for rendering EMR data and language interpretation data on disparate devices at a healthcare provider location.

### DETAILED DESCRIPTION

To provide for a more seamless and efficient user interface experience, a computer-implemented process allows for an EMR device to initiate a language interpretation request at a healthcare provider location and a language interpretation device to provide language interpretation services, based upon the request, also at the healthcare provider location. (The nomenclature “EMR device” is referred to herein as any computing device that is being used by a healthcare provider to access/manage EMR records during a patient visit, and the nomenclature “language interpretation device” is referred to herein as any computing device that is being used by the healthcare provider to obtain language interpretation services for the healthcare provider and/or the LEP during the patient visit.) The healthcare provider is able to continue updating the EMR during the patient visit without interference of the language interpretation session being rendered by the user interface on the EMR device. Accordingly, the healthcare provider is able to invoke a language interpretation session from the EMR device, link the language interpretation device, and fulfill the language interpretation service request at the language interpretation device while simultaneously interacting with the EMR data on the user interface of the EMR device.

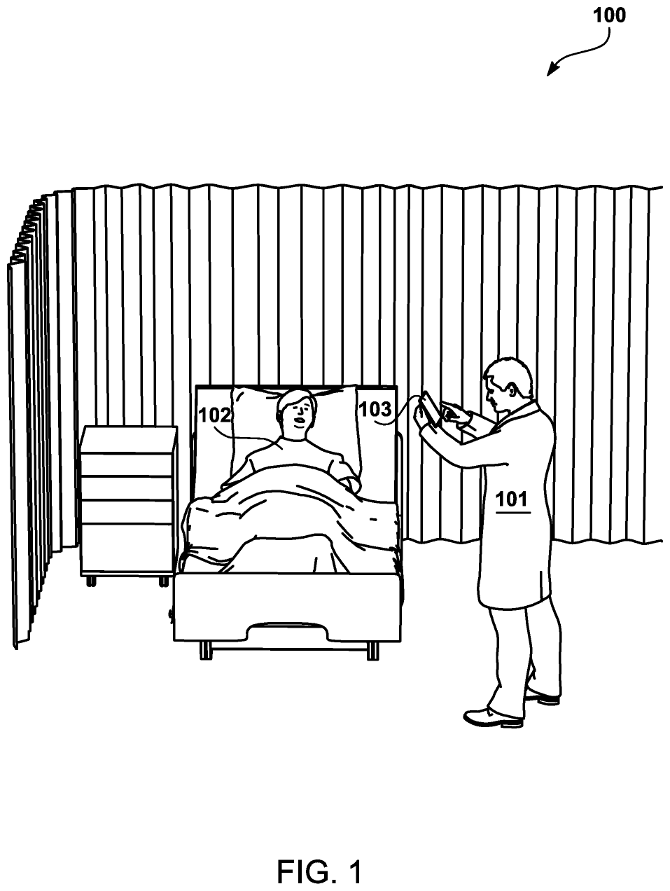

FIG. 1 illustrates a healthcare provider location 100 (e.g., a doctor's office, health clinic, dentist's office, etc.) in which a healthcare provider 101 provides in-person healthcare services to an LEP patient 102. To help facilitate such services, the healthcare provider 101 uses an EMR device 103 (e.g., tablet device) to review and/or update EMR records of the LEP patient 102 during the office visit. (Although a tablet device is illustrated, other types of EMR devices 103 (e.g., desktop computer, mobile phone, etc.) may be utilized by the healthcare provider 101.)

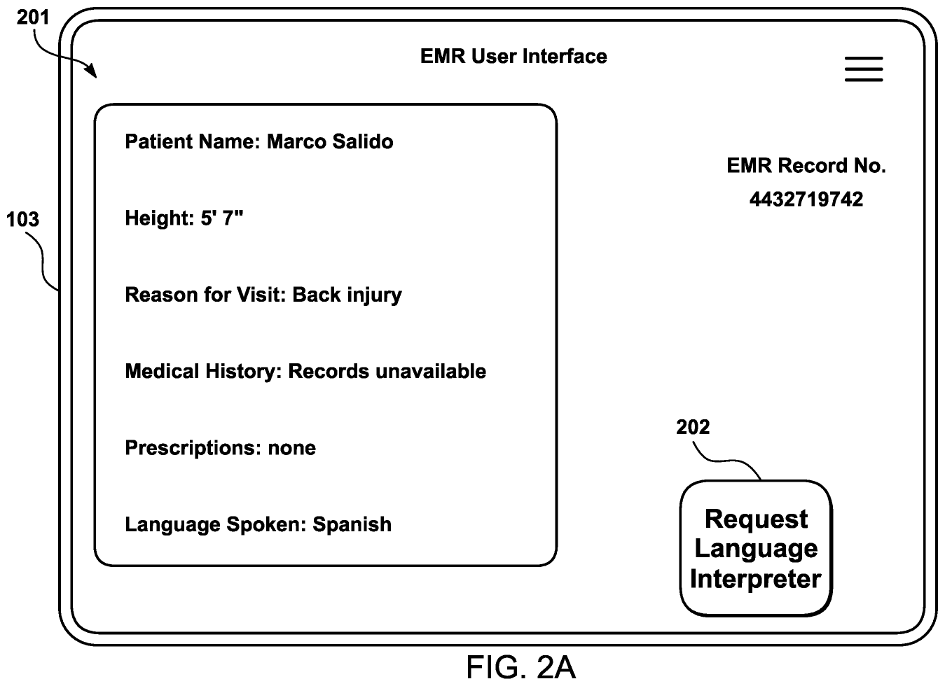

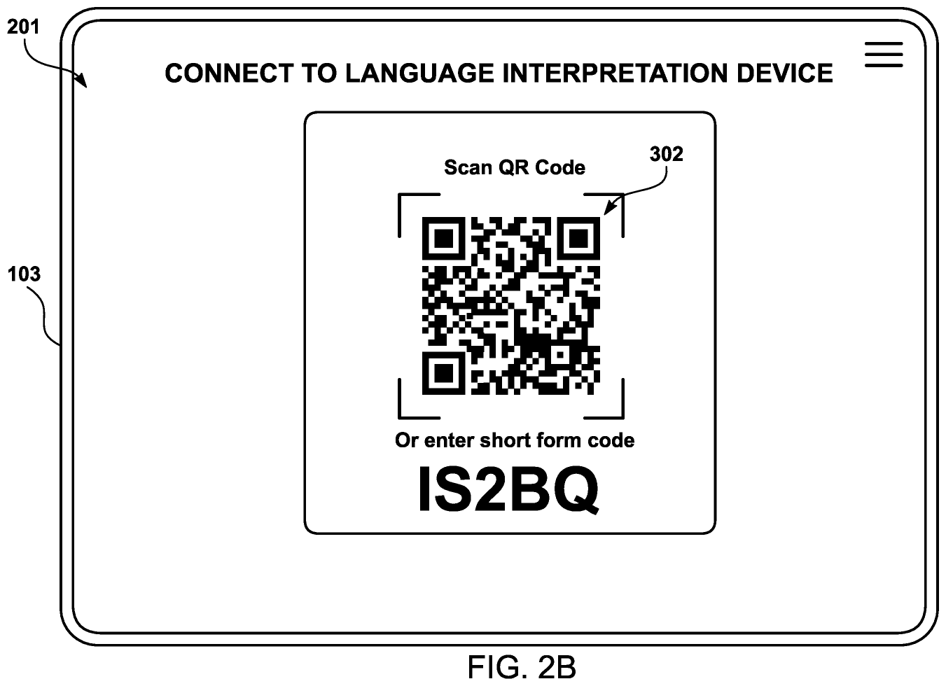

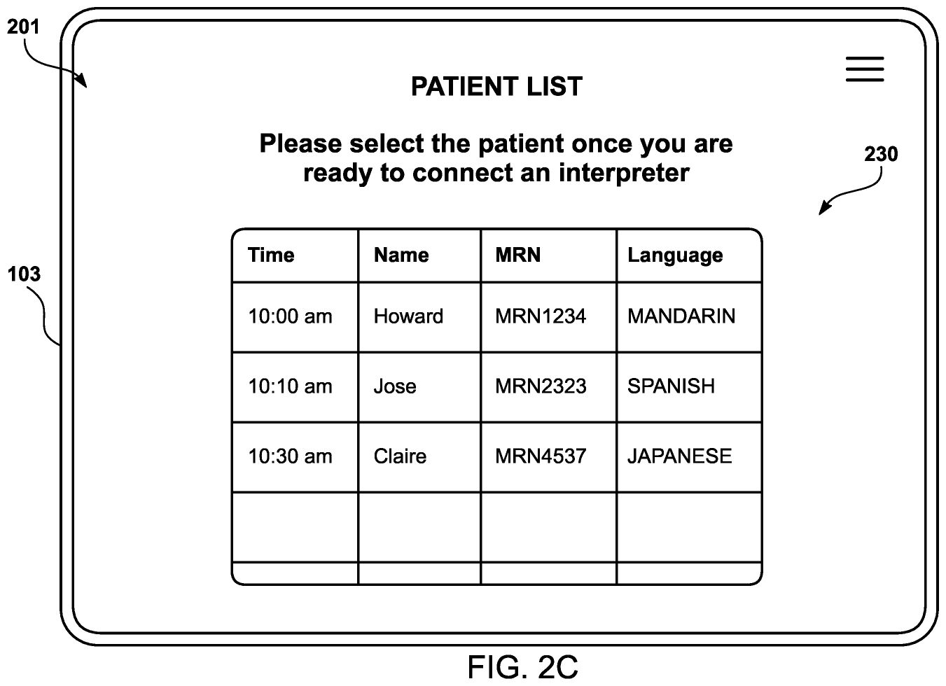

As an example, FIG. 2A illustrates an EMR user interface 201 that may be rendered by the EMR device 103. For instance, the EMR user interface 201 may render data such as the EMR number (or other identifier), patient name, height, weight, date of birth, reason for office visit, medical history, prescriptions, etc. (The EMR user interface 201 may include additional pages/fields not shown to allow the healthcare provider 101 to update the medical chart of the LEP patient 102.) Additionally, the EMR user interface 201 may indicate a language spoken by the LEP patient 102. (As an example, Spanish is depicted, but other languages may be depicted instead.)

Furthermore, the EMR user interface 103 has a language interpretation request indicium 202. When activated by a user input from the healthcare provider 101, the language interpretation request indicium 202 may be used to place a request for a language interpretation service to be rendered on a separate device (i.e., a language interpretation device). In essence, the EMR session will be linked on two different devices, but allow for the healthcare provider 101 to use the EMR device 103 without the interference of a language interpretation software application. As a result, the healthcare provider 101 is able to seamlessly update and manage EMR records during the patient visit.

FIG. 2B illustrates an example of a QR code and a short form code (e.g., a set of alphanumeric letters, digits, or the like) being displayed by the EMR device 103 for image capture by the language interpretation device 301. In effect, the healthcare provider 101 is given the choice to scan a QR code from the EMR device 103 with a language interpretation device 301 or enter the short form code. For example, scanning a QR code may be impractical when the healthcare provider 101 is using a desktop computer; in which case, the short form code may be entered.

FIG. 2C illustrate an alternative process in which the healthcare provider 101 may link the EMR device 103. For instance, a table 230 listing patients may be displayed so that the healthcare provider 101 may click on an indicium (e.g., a row, patient name, etc.) to transfer date from the EMR device 103 to the language interpretation device 301. As an example, the table 230 may be utilized if the healthcare provider 101 is having difficulty performing the linking using a QR code or short form code.

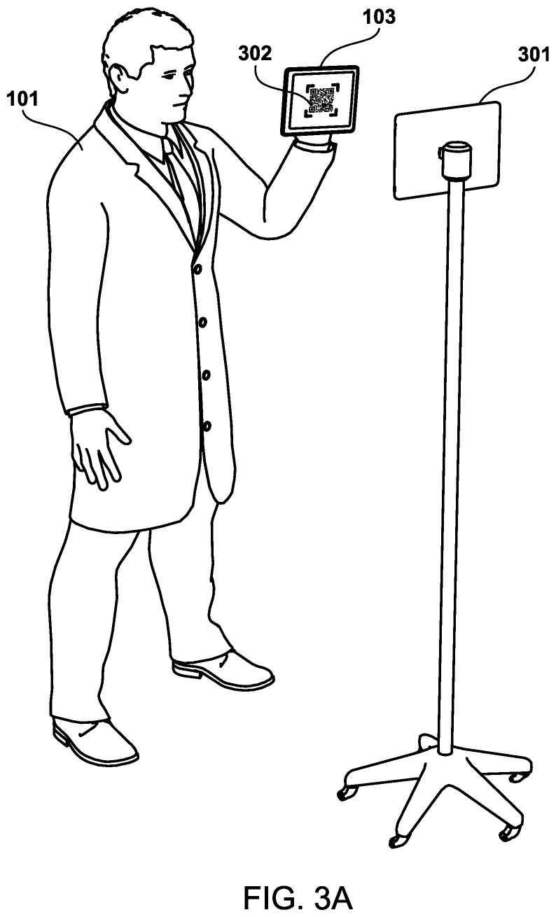

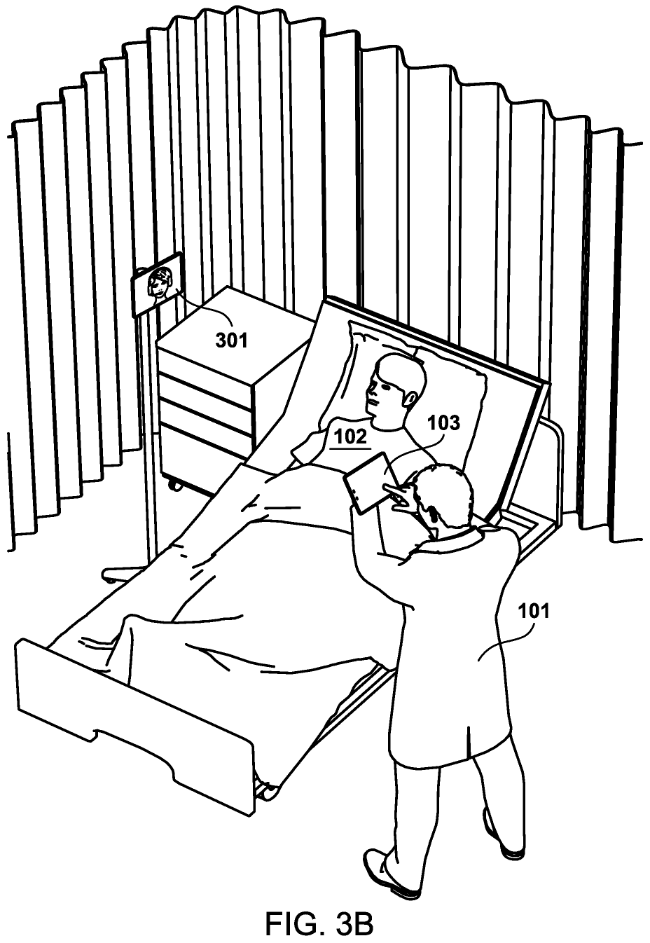

FIG. 3A illustrates an example of how the EMR device 103 is linked with a language interpretation device 301. (An example of the language interpretation device 301 as a tablet device positioned on a mobile stand configuration is just one of a variety of possible language interpretation devices 301.) In particular, a visual code indicium (e.g., QR code, short form identifier, etc.) is displayed by the EMR device 103. The language interpretation device 301 may capture an image (e.g., via an image capture device) of the visual indicium. Alternatively, the healthcare provider 101 may provide a manual input of the visual code indicium 302. The visual code indicium 302 may include one or more of the following identifiers: patient identification information, office visit information, healthcare provider information, and EMR device 103 information. In one embodiment, the foregoing information is securely encrypted. Accordingly, the EMR data that is being accessed on the EMR device 103 may be transmitted to the language interpretation device 301. As a result, the EMR device 103 is able to invoke a language interpretation request and pass information to the language interpretation device 301 to allow the language interpretation device 301 to render language interpretation services in a dedicated manner.

FIG. 3B illustrates the healthcare provider 101 and the LEP patient 102 participating in a language interpretation session via the language interpretation device 301. For instance, a virtual language interpreter 301 may be rendered by the language interpretation device 301. Accordingly, the healthcare provider 101 is able to interact with the EMR device 103 while also participating in a language interpretation session with the LEP patient 102 via the language interpretation device 103.

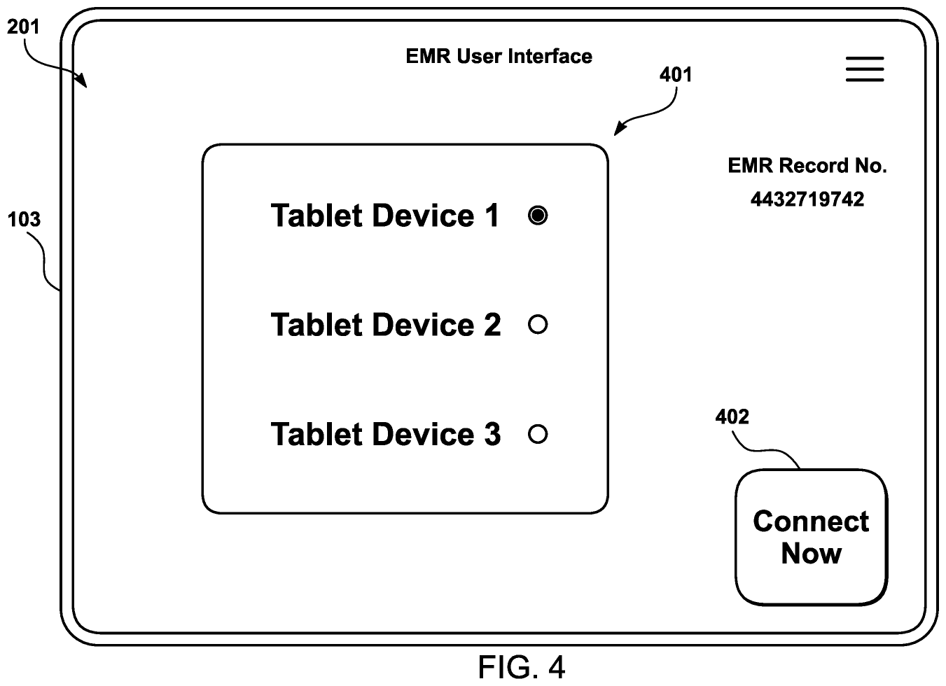

Furthermore, a plurality of different language interpretation devices may be linked to the EMR device 103. FIG. 4 illustrates the EMR user interface 201 of the EMR device 103 rendering a language interpretation device menu 401 of pre-linked language interpretation devices. Accordingly, the healthcare provider 101 may seamlessly move from room to room and select the language interpretation device that is mostly proximately positioned. In another embodiment, the list of available language interpretation devices 103 is automatically populated based on those that are available via a computerized network, such as a wireless network. A connect indicium 402 may be utilized to finalize the selection.

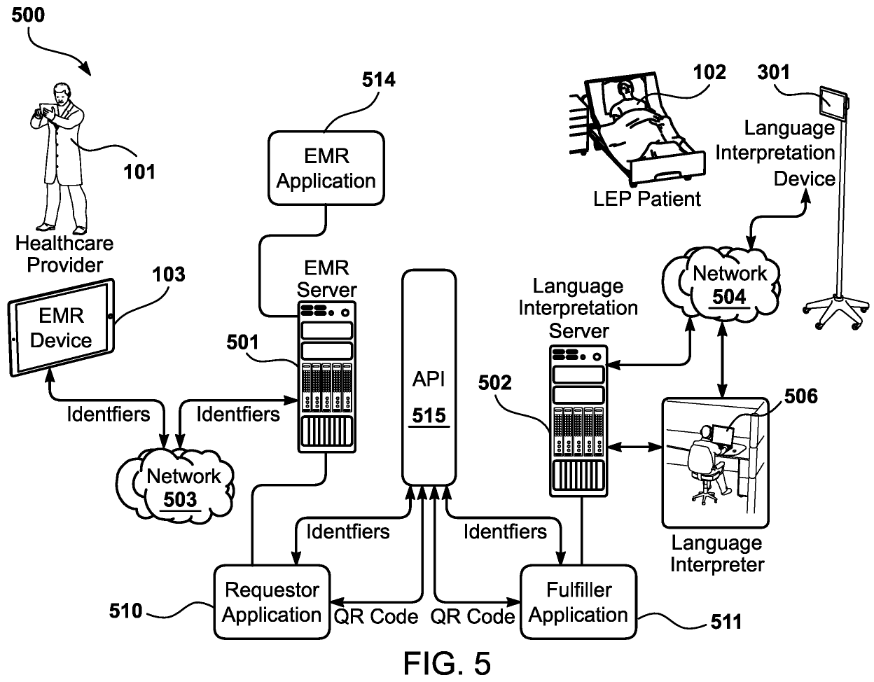

FIG. 5 illustrates a requestor/fulfiller configuration 500 that links the EMR device 103 with the language interpretation device 301. In particular, the EMR device 103 may operate a cloud-based EMR application 514 that allows for access and modification of EMR data stored by an EMR server 501 (e.g., via database of EMRs) through a computerized network 503. For instance, the EMR device 103 may obtain an EMR for rendering via the EMR user interface 201 illustrated in FIG. 2. Additionally, the EMR device 103 may operate a requestor application 510 via the EMR sever 501. (In one embodiment, the requestor application 510 and the EMR application 514 are two different software applications. In another embodiment, they are integrated into the same software application.) For instance, the language interpretation request indicium 202 of the EMR user interface 201 may invoke the requestor application 510. In particular, the requestor application 510 may receive one or more of the identifiers (e.g., patient identification information, office visit information, healthcare provider information, and EMR device 103 information). The requestor software application 510 will then queue the identifiers using an application programming interface (“API”) 515. Upon receipt of the identifiers from the API 515, the fulfiller application 511 may generate a QR code (or other visual code indicium 302) to be sent via the API 515 to the requestor application 510. In essence, the fulfiller application 511 is able to communicate with a language interpretation server 502 to request the services of a language interpreter 505 who speaks both the language of the healthcare provider 101 and the patient LEP 301. After the establishment of the language interpretation session, the fulfiller application 511 has information regarding the language interpretation session and the EMR device 103. Accordingly, the QR code generated by the fulfiller application 511 may include the identifiers and language interpretation session data. Based on information contained in the QR code, the language interpretation device 301 is able to link to the language interpretation session. As a result, the patient information may be sent to the language interpretation device 301 to allow for synchronized, simultaneous usage of the patient data by both the EMR device 103 and the language interpretation device 301. Thus, the EMR device 103 is able to be used independently of the language interpretation device 301. Furthermore, the language interpretation service may be tied directly to a particular EMR, thereby allowing for billing and reimbursement processing by the language interpretation service provider particular to a given healthcare provider 101 and/or LEP patient 102.

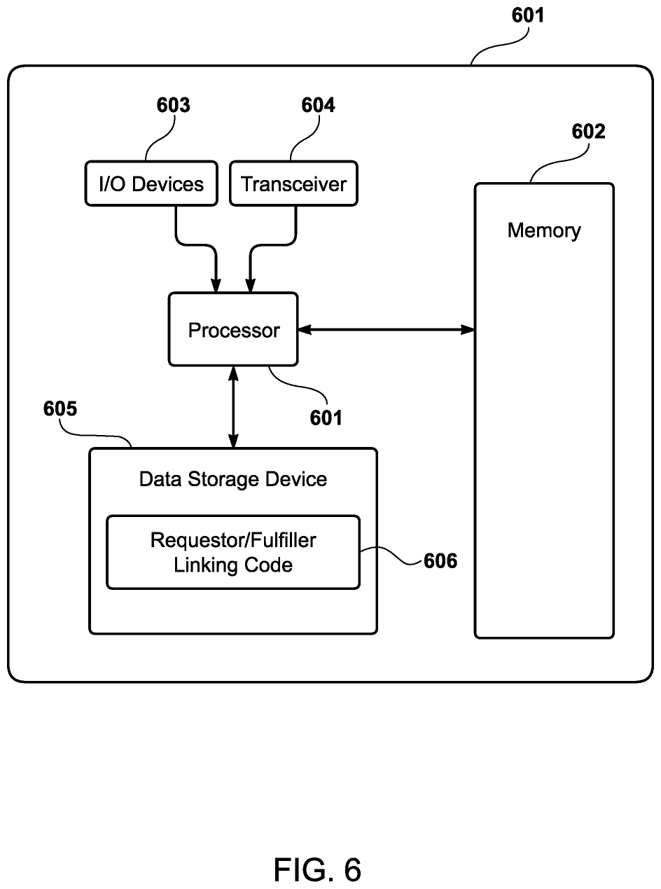

FIG. 6 illustrates a system configuration for the requestor/fulfiller configuration 500 illustrated in FIG. 5. A processor 601 may be specialized for requestor/fulfiller operations. The system configuration may also include a memory device 602, which may temporarily store data linking the EMR device 103 to the language interpretation device 301. For example, a requestor/fulfiller data structure may be composed to store data pertaining to the linkage. The requestor/fulfiller data structure improves the usability of the EMR user interface 201 by allowing the EMR device 103 be used independently of the language interpretation device 301. Furthermore, the memory device 602 may temporarily store computer readable instructions performed by the processor 601. As an example of such computer readable instructions, a data storage device 605 within the system configuration may store requestor/fulfiller linking code 606 that may be utilized to compose, maintain, and access the requestor/fulfiller data structure. Various devices (e.g., keyboard, microphone, mouse, pointing device, hand controller, joystick, display screen, holographic projector, etc.) may be utilized for input/output (“I/O”) devices 603. The system configuration may also have a transceiver 604 to send and receive data. Alternatively, a separate transmitter and receiver may be used instead.

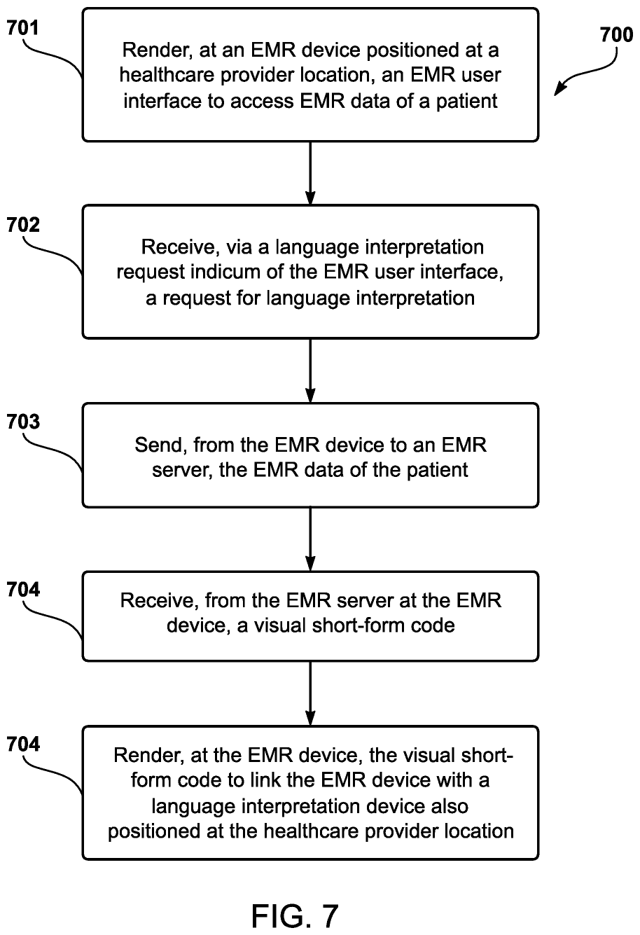

FIG. 7 illustrates a computer-implemented process 700 for rendering EMR data and language interpretation data on disparate devices at a healthcare provider location. At a process block 701, the computer-implemented process 700 renders, at an EMR device 103 positioned at a healthcare provider location 100, an EMR user interface 201 to access EMR data of a patient. Furthermore, at a process block 702, the computer-implemented process 700 receives, via a language interpretation request indicium 202, a request for language interpretation. At a process block 703, the computer-implemented process 700 sends, from the EMR device 103 to the EMR server 501, the EMR data of the patient. Additionally, at a process block 704, the computer-implemented process receives, from the EMR server 501 at the EMR device 103, a visual code indicium. Finally, at a process block 705, the computer-implemented process 700 renders, at the EMR device 103, the visual code indicium to link the EMR device 103 with a language interpretation device 301 also positioned at the healthcare provider location 100 such that the language interpretation device 103 renders a language interpretation session between a healthcare provider 101 and the patient 102 without being rendered by the EMR device 103.

It is understood that the apparatuses, systems, computer program products, and processes described herein may also be applied in other types of apparatuses, systems, computer program products, and processes. Those skilled in the art will appreciate that the various adaptations and modifications of the embodiments of the apparatuses, systems, computer program products, and processes described herein may be configured without departing from the scope and spirit of the present apparatuses, systems, computer program products, and processes. Therefore, it is to be understood that, within the scope of the appended claims, the present apparatuses, systems, computer program products, and processes may be practiced other than as specifically described herein.

We claim:

1. A computer-implemented process comprising:
rendering, at an EMR device positioned at a healthcare provider location, an EMR user interface to access EMR data of a patient;
receiving, via a language interpretation request indicum of the EMR user interface, a request for language interpretation;
sending, from the EMR device to an EMR server, the EMR data of the patient;
receiving, from the EMR server at the EMR device, a visual code indicium; and
rendering, at the EMR device, the visual code indicium to link the EMR device with a language interpretation device also positioned at the healthcare provider location such that the language interpretation device renders a language interpretation session between a healthcare provider and the patient without being rendered by the EMR device.

2. The computer-implemented process of claim 1, wherein the EMR server communicates with a language interpretation server via an API.

3. The computer-implemented process of claim 1, wherein the visual code indicium is a QR code.

4. The computer-implemented process of claim 1, wherein the EMR user interface renders a menu of linked language interpretation devices from which the language interpretation device is selected via a user input.

5. The computer-implemented process of claim 1, wherein the EMR user interface renders a menu of automatically populated language interpretation devices, available via a wireless network, from which the language interpretation device is selected via a user input.

6. The computer-implemented process of claim 1, wherein the language interpretation server accesses the EMR data to manage accounting for the language interpretation session.

7. The computer-implemented process of claim 1, further comprising accessing a requestor/fulfiller data structure that links the EMR device to the language interpretation device.

8. A computer program product comprising a computer readable storage device having a computer readable program stored thereon, wherein the computer readable program when executed on a computer causes the computer to:
render, at an EMR device positioned at a healthcare provider location, an EMR user interface to access EMR data of a patient;
receive, via a language interpretation request indicum of the EMR user interface, a request for language interpretation;
send, from the EMR device to an EMR server, the EMR data of the patient;
receive, from the EMR server at the EMR device, a visual code indicium; and
render, at the EMR device, the visual code indicium to link the EMR device with a language interpretation device also positioned at the healthcare provider location such that the language interpretation device renders a language interpretation session between a healthcare provider and the patient without being rendered by the EMR device.

9. The computer program product of claim 8, wherein the EMR server communicates with a language interpretation server via an API.

10. The computer program product of claim 8, wherein the visual code indicium is a QR code.

11. The computer program product of claim 8, wherein the EMR user interface renders a menu of linked language interpretation devices from which the language interpretation device is selected via a user input.

12. The computer program product of claim 8, wherein the EMR user interface renders a menu of automatically populated language interpretation devices, available via a wireless network, from which the language interpretation device is selected via a user input.

13. The computer program product of claim 8, wherein the language interpretation server accesses the EMR data to manage accounting for the language interpretation session.

14. The computer program product of claim 8, wherein the computer is further caused to access a requestor/fulfiller data structure that links the EMR device to the language interpretation device.

15. A computer-implemented system comprising:
a language interpretation device that establishes a virtual language interpretation session with a remote language interpreter; and
an EMR device positioned at a healthcare provider location, the EMR device rendering an EMR user interface to access EMR data of a patient, receiving, via a language interpretation request indicum of the EMR user interface, a request for language interpretation, sending the EMR data of the patient to an EMR server, receiving a visual code indicium, and rendering the visual code indicium to link the EMR device with a language interpretation device also positioned at the healthcare provider location such that the language interpretation device renders a language interpretation session between a healthcare provider and the patient without being rendered by the EMR device.

16. The computer-implemented system of claim 15, wherein the EMR server communicates with a language interpretation server via an API.

17. The computer-implemented system of claim 15, wherein the visual code indicium is a QR code.

18. The computer-implemented system of claim 15, wherein the EMR user interface renders a menu of linked language interpretation devices from which the language interpretation device is selected via a user input.

19. The computer-implemented system of claim 15, wherein the EMR user interface renders a menu of automatically populated language interpretation devices, available via a wireless network, from which the language interpretation device is selected via a user input.

20. The computer-implemented system of claim 15, wherein the language interpretation server accesses the EMR data to manage accounting for the language interpretation session.
